(19) United States
(12) Patent Application Publication
(10) Pub. No.: US 2026/0002834 A1
(43) Pub. Date: Jan. 1, 2026

(54) GLASSES DIOPTER IDENTIFICATION METHOD AND APPARATUS, ELECTRONIC DEVICE AND STORAGE MEDIUM

(71) Applicant: Beijing Zitiao Network Technology Co., Ltd., Haidian District, Beijing (CN)

(72) Inventor: Xiang LONG, Beijing (CN)

(21) Appl. No.: 18/881,918

(22) PCT Filed: Nov. 21, 2023
(86) PCT No.: PCT/CN2023/132905

§ 371 Date: Jan. 7, 2025

(30) Foreign Application Priority Data
Dec. 5, 2022 (CN) 202211551898.2

(51) Int. Cl. G01M 11/02; G06T 7/149

(52) U.S. Cl. CPC G01M 11/0228; G01M 11/0264; G06T 7/149; G06T 2207/20081

(57) ABSTRACT

The present disclosure relates to a glasses diopter identification method and apparatus, an electronic device, and a storage medium, the method includes: acquiring an image to be processed; the image to be processed is obtained by performing image acquisition on a lens of target glasses under irradiation of a light source, and the image to be processed includes an optical feature generated when the light source emits light to the lens of the target glasses; and inputting input data including the image to be processed to a glasses diopter identification model to obtain a glasses diopter of the target glasses.

### CROSS REFERENCE TO RELATED APPLICATIONS

The present application is based on and claims priority from CN patent application Ser. No. 202211551898.2 filed on Dec. 5, 2022, the disclosure of which is incorporated by reference herein in its entirety.

### TECHNICAL FIELD

The present disclosure relates to the field of computer technologies, and in particular, to a glasses diopter identification method and apparatus, an electronic device, and a storage medium.

### BACKGROUND

Because the refraction effects brought by the glasses with different diopters are completely different, the eye movement interaction algorithm needs to acquire the glasses diopters to correspondingly correct the algorithm result, so that the identification of the glasses diopters is very important for eye movement interaction.

### SUMMARY

This disclosure provides a glasses diopter identification method and apparatus, an electronic device, and a storage medium.

According to one aspect of the present disclosure, a glasses diopter identification method is provided, the method includes:
acquiring an image to be processed; the image to be processed is obtained by performing image acquisition on a lens of target glasses under irradiation of a light source, and the image to be processed includes an optical feature generated when the light source emits light to the lens of the target glasses; and
inputting input data including the image to be processed to a glasses diopter identification model to obtain a glasses diopter of the target glasses.

According to another aspect of the present disclosure, a glasses diopter identification apparatus is provided, the apparatus includes:
an image acquisition module configured to acquire an image to be processed; the image to be processed is obtained by performing image acquisition on a lens of target glasses under irradiation of a light source, and the image to be processed includes an optical feature generated when the light source emits light to the lens of the target glasses; and
a glasses diopter identification module configured to input data including the image to be processed to a glasses diopter identification model to obtain a glasses diopter of the target glasses.

According to another aspect of the present disclosure, an electronic device is provided. The electronic device includes: memory and processor, the memory stores computer program, and the processor executes the program to implement the above method.

According to another aspect of the present disclosure, a computer-readable storage medium is provided, the computer-readable storage medium stores computer program thereon, and the program, when executed by a processor to implement the above method.

### BRIEF DESCRIPTION OF THE DRAWINGS

Further details, features and advantages of the disclosure are disclosed in the following description of exemplary embodiments, taken in conjunction with the accompanying drawings, in which:

FIG. 1 is a schematic diagram of a VR device provided in an exemplary embodiment of the present disclosure;

FIG. 2A is an effect diagram of a first lens obtained by performing image acquisition for the target glasses according to an exemplary embodiment of the present disclosure;

FIG. 2B is an effect diagram of a second lens obtained by performing image acquisition for the target glasses according to an exemplary embodiment of the present disclosure;

FIG. 3 is a flowchart of a glasses diopter identification method according to an exemplary embodiment of the present disclosure;

FIG. 4 is another flowchart of the glasses diopter identification method according to an exemplary embodiment of the present disclosure;

FIG. 5 is another flowchart of the glasses diopter identification method according to an exemplary embodiment of the present disclosure;

FIG. 6 is a schematic diagram of the glasses diopter identification apparatus according to an exemplary embodiment of the present disclosure;

FIG. 7 is a structural block diagram of an electronic device according to an exemplary embodiment of the present disclosure;

FIG. 8 is a structural block diagram of a computer system according to an exemplary embodiment of the present disclosure.

### DETAILED DESCRIPTION

Embodiments of the present disclosure will be described in more detail below with reference to the accompanying drawings. While certain embodiments of the present disclosure are shown in the drawings, it should be understood that the present disclosure may be embodied in various forms and should not be construed as limited to the embodiments set forth herein, but rather are provided for a more complete and thorough understanding of the present disclosure. It should be understood that the drawings and the embodiments of the disclosure are for illustration purposes only and are not intended to limit the scope of the disclosure.

It should be understood that the various steps recited in method embodiments of the present disclosure may be performed in a different order, and/or performed in parallel. Moreover, method embodiments may include additional steps and/or omit performing the illustrated steps. The scope of the present disclosure is not limited in this respect.

The term “including” and variations thereof as used herein is intended to be open-ended, i.e., “including but not limited to”. The term “based on” is “based at least in part on”. The term “one embodiment” means “at least one embodiment”; the term “another embodiment” means “at least one additional embodiment”; the term “some embodiments” means “at least some embodiments”. Relevant definitions for other terms will be given in the following description. It should be noted that the terms “first”, “second”, and the like in the present disclosure are only used for distinguishing different devices, modules or units, and are not used for limiting the order or interdependence of the functions performed by the devices, modules or units.

It is noted that references to “a”, “an” or “a plurality of” in this disclosure are intended to be illustrative rather than limiting, and that those skilled in the art will appreciate that references to “one or more” are intended to be exemplary and not limiting unless the context clearly indicates otherwise.

The names of messages or information exchanged between devices in the embodiments of the present disclosure are for illustrative purposes only, and are not intended to limit the scope of the messages or information.

When the glasses diopter is measured, a user is usually required to go to a special testing organization to detect the glasses diopter through a special optical instrument, so that the measurement cost of the glasses diopter is higher. Because the refraction effects brought by the glasses with different diopters are completely different, the glasses diopters are required to be obtained to correspondingly correct the algorithm result when the eye movement interaction algorithm is adopted, and therefore, the identification of the glasses diopters is very important for the eye movement interaction.

When the eye movement interaction algorithm is adopted, two aspects of correction are needed to be carried out on the algorithm result through the glasses diopter: firstly, a glasses lens is added between a camera and human eye, so that a certain offset exists between an observed sight line direction and a real sight line direction of the eyes, the offset is larger when the diopter is larger, and the near vision and the far vision have offset effects in different directions; secondly, a glasses lens is added between the camera and the human eye, so that a certain difference exists between the observed pupil distance and the real pupil distance, the difference is related to the diopter of glasses and the position of the glasses, and systematic rough correction can be performed by using the glasses diopter.

The eye movement interaction algorithm is widely applied to VR (Virtual Reality) device, the value of diopters that glasses are worn by a user is accurately and quickly identified, and the eye movement interaction algorithm can be effectively performed on the VR device by correcting according to the value of glasses diopters, so that better experience result is brought to the user by the VR device.

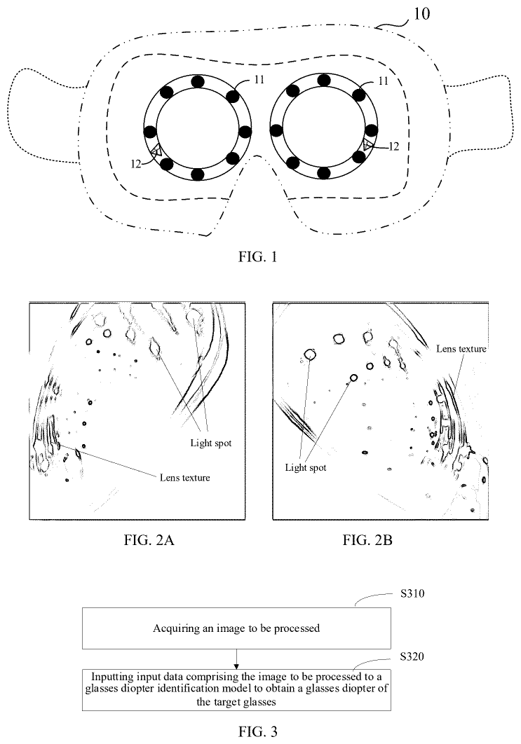

Therefore, in order to accurately, quickly and effectively perform glasses diopter identification for the glasses worn by the user, in the embodiment provided by the present disclosure, as shown in FIG. 1, at least one light source 11 and one image acquisition device 12 are respectively disposed on two lens barrels of the VR device 10. The image acquisition device may be a camera, the camera may be an infrared camera, and the light source may be an infrared light source, for example, a Light Emitting Diode (LED for short). In the embodiments, an LED lamp as a light source is taken as an example for description, and the LED lamp related in the embodiments may be an infrared LED lamp, or may also be a common LED lamp, which may be specifically selected according to the needs.

It should be noted that, in the image acquisition device in the embodiment of the present disclosure, the operating wavelength band should be matched with the wavelength band of the light emitted by the light source. For example, the light source in the embodiment may be an infrared light source, and since infrared light is not perceivable by human eyes, the human eyes are not easily injured, and normal display of the VR device is not affected. In addition, the image acquisition device may also be a device that performs imaging based on infrared light.

In this way, when the user uses the VR device by wearing it, by opening a plurality of LED lamps that set up respectively on two lens barrels, these LED lamps can emit light to the area in which the glasses located, and light can produce reflection light spots and lens texture on the lens of glasses. As shown in FIGS. 2A and 2B, FIG. 2A is an effect image of the first lens of the target glasses, and FIG. 2B is an effect image of the second lens of the target glasses.

Therefore, when detecting that the user is wearing the VR device, the embodiment of the present disclosure can turn on the LED lamps on the two lens barrels, and turn on the cameras on the two lens barrels to perform image acquisition. Since the light emitted by the LED lamp may generate optical features on the lenses of the glasses worn by the user, for example, reflection light spots and glasses textures are generated on the lenses, the image captured by the image acquisition device may generally include the light spots and the glasses textures. In addition, the light emitted by the LED lamp may generate phenomena such as diffuse reflection on the side surface of the lens, and the image acquisition device may also capture the thickness of the lens.

In an embodiment, the glasses diopter of the myopia glasses and the glasses diopter of the hyperopia glasses are different, because the lenses with different glasses diopters have different configurations, the optical features generated on the lenses with different diopters by the light emitted by the light source are also different, that is, the optical features generated on the lenses by the light emitted by the light source can reflect the glasses diopters. The glasses diopter of the glasses can be identified through the optical features generated on the lenses by the light emitted by the light source. The optical features generated on the lens by the light emitted by the light source can include one or more of light spots, lens textures and lens thickness information.

Specifically, in general, two groups of distinct reflection light spots are formed on the lens of the glasses by the LED lamp, one group is the reflection light spots formed by specular reflection on the outer surface of the lens of the glasses, and the other group is the reflection light spots formed on the inner surface of the lens of the glasses. The positions and the sizes of the light spots on the inner surface and the outer surface are determined by the curvatures of the inner surface and the outer surface of the lens of the glasses, and the curvatures of the two curved surfaces also determine the diopter of the glasses. Therefore, the light spot on the lens can reflect the glasses diopter of the glasses.

In the embodiment, when light is emitted to the lens of the glasses by the LED lamp, the light is generally reflected and refracted for multiple times inside the lens to generate a ring-shaped texture, i.e., a lens texture. Thus, the texture on the lens may also reflect the glasses diopter of the glasses. Generally, the higher the glasses diopter, when the camera takes the pictures of the glasses, the more the portion of the glasses lens near the edge will present a circle of texture, and these textures can be used to estimate the glasses diopter. In the embodiment, the glasses diopter of the glasses may be a myopia diopter or a hyperopia diopter. Of course, the glasses can also be plane lens, such as blue light prevention glasses which are worn by users at ordinary times and have no diopter.

In general, the higher the value of glasses diopters, the thicker the frame, and the camera captures the thickness information of the frame even when capturing the glasses. Therefore, the thickness information of the lens can also reflect the glasses diopter of the glasses.

It should be noted that, when a camera captures the lenses from the side, the lens texture and the lens thickness with better capturing result can be obtained, so a certain non-vertical intersection angle between the camera and the lens may be required. Certainly, because the lenses of the common glasses are all curved surfaces, when a camera on a lens barrel of the VR device collects images of the glasses, a certain non-vertical intersection angle is formed between the lenses and the lenses, and the collection of the information such as light spots, textures and thicknesses of the lenses is not influenced.

Therefore, in the embodiment of the present disclosure, images of two lenses of glasses may be respectively collected through cameras on two lens barrels of the VR device, and collected image information may be input into a trained glasses diopter identification model to identify the glasses diopter, so as to obtain the glasses diopter of the glasses, where the glasses diopter includes the diopters of the two lenses of the glasses.

In the embodiment, images of two lenses of the glasses, namely a first lens image and a second lens image, can be respectively collected by cameras on two lens barrels of the VR device. In some examples, the first lens image and the second lens image may be used as images to be processed, respectively, and as an input of the glasses diopter identification model. In other examples, the images of the two lenses may be spliced in the horizontal direction, and the spliced image may be used as an input of the glasses diopter identification model. After the first lens image and the second lens image are respectively used as images to be processed and input into the glasses diopter identification model, the glasses diopters of the first lens and the second lens can be respectively identified. The images of the two lenses are spliced in the horizontal direction, and the spliced images are used as the input of the glasses diopter identification model, so that the glasses diopters of the two lenses of the glasses can be obtained simultaneously. Since there is a certain correlation between the glasses diopters of the left and right eyes of a human body in general, when the first lens image and the second lens image are spliced and the spliced image is input to the glasses diopter identification model, a more accurate identification effect can be achieved. Correspondingly, during the training process of the glasses diopter identification model, the spliced images can be used as training samples, so that the model can learn the incidence relation between the images of the two lenses of the glasses in the training process, and the trained model can better identify the glasses diopters of the glasses.

In the embodiments provided by the present disclosure, when the VR device is worn on the eyes of the user, the image content included in the captured image thereof is different according to the pose, the field of view, and the like of the image acquisition device itself. For example, in some examples, the image content in the first and second lens images captured by the image acquisition device may include only the lens area. In other examples, the image content in the first lens image and the second lens image captured by the image acquisition device may include both the lens area and the area outside the lens, where the area outside the lens may be other facial areas of the user (e.g., nose, forehead, etc.). Since the image acquisition device emits light to the area where the target glasses are located during the capturing process, the lens areas in the first lens image and the second lens image captured by the image acquisition device may include optical features, such as light spot information. Therefore, in order to enable the glasses diopter identification model to concentrate more on the glasses-related information in the input data, thereby extracting more effective information for identifying the glasses diopter and ensuring the identification efficiency, in the embodiment, the image segmentation model may further perform image segmentation on the first lens image and the second lens image to obtain the lens image and the light spot image of the first lens image and the lens image and the light spot image of the second lens image, respectively. The lens image segmented from the first lens image and the lens image segmented from the second lens image may only include the lens area. Then the lens image, the light spot image and the first lens image which are segmented from the first lens image are spliced in a channel dimension to form a first lens spliced image; and simultaneously the lens image, the light spot image and the second lens image which are segmented from the second lens image are spliced in the channel dimension to form a second lens spliced image. The first lens spliced image and the second lens spliced image can be respectively used as input of the glasses diopter identification model, and the glasses diopter of the first lens and the glasses diopter of the second lens are respectively identified. The first lens spliced image and the second lens spliced image can be spliced in the horizontal direction and used as input of the glasses diopter identification model to identify the glasses diopter of the first lens and the glasses diopter of the second lens. The glasses diopter identification model in the embodiment of the present disclosure may be a depth regression model after training. For example, if the first lens image is an RGB color image, the first lens image may contain R, G, B channels, and the segmented lens image, the light spot image, and the first lens image are spliced together in the channel dimension, so that the spliced first lens image has five channels. If the first lens image is captured through the infrared camera, the first lens image only has one channel, the segmented lens image, the light spot image and the first lens image are spliced in the channel dimension, and the spliced first lens image has three channels.

It should be noted that, because the light spots, the lens textures, and the lens thickness information on the lenses can all reflect the value of glasses diopters, in the embodiment, when identifying the value of glasses diopters, at least one of the information including the light spots, the lens textures, and the lens thickness information can be used as an input of the glasses diopter identification model, so that the identification of the value of glasses diopters can be realized. The images containing the light spots, the lens textures and the lens thickness information can be used as input of the glasses diopter identification model to identify the glasses diopter, so that the glasses diopter can be identified more accurately. In the training process of the glasses diopter identification model, the adopted training sample can be a sample image containing at least one of light spots, lens textures and lens thickness information, the sample image can be marked with a light spot area, a lens area and/or lens thickness information, and the sample can also carry the corresponding glasses diopter.

In an embodiment, during the process of segmenting the light spots from the first lens image and the second lens image, an image segmentation model may be used for processing. The image segmentation model can be obtained by training a preset model with a training sample, and the training sample can include an image acquired by an image acquisition device on VR when a user is wearing glasses. In addition, in order to obtain sufficient samples and reduce the acquisition cost of the training samples, a plurality of training samples can be obtained by image synthesis. The thickness information of the lens can be detected by identifying the edge of the lens, which is not described herein again.

Specifically, in the embodiment, various types of glasses can be collected, so that the subject wears a plurality of different pairs of glasses, and the eye image of the subject is captured by using a camera on the VR device. Each pair of glasses needs to be captured at various wearing positions, while the lens area and the light spot area of the glasses are marked. For example, the light spot areas may be marked manually, or the image may be binarized by using a threshold algorithm, and an area having a pixel value greater than a threshold value is used as the light spot area.

In the embodiment, a camera on the VR device can be used for capturing only glasses images to obtain a large number of different images containing glasses textures, and the lenses area and the reflect light spot area of the glasses are marked. The eye image data of a large number of subjects without glasses are collected, the eye image data and the glasses images of the subjects are combined randomly by using an image synthesis method, the synthesized data with glasses are generated in batch, and the synthesized data are used as training samples of the image segmentation model, so that sufficient training samples can be obtained to train the model, and the acquisition cost of the sample is greatly reduced. In addition, the image segmentation model after training can be finely adjusted through non-synthesized sample data, so that the acquisition cost of the training sample is reduced by taking the synthesized data as the training sample, and the processing effect of the model on real data can be ensured by finely adjusting the image segmentation model after training with the non-synthesized data. Non-synthesized data refers to the data directly collected by the image acquisition device set on the VR device after the subject wears different types of glasses.

In combination with the above embodiment, in a further embodiment provided by the present disclosure, an embodiment of the present disclosure further provides a method for identifying a glasses diopter, as shown in FIG. 3, the method may include the following steps:
in step S310, acquiring an image to be processed.

The image to be processed is obtained by performing image acquisition on the lens of the target glasses under the irradiation of the light source, and the image to be processed includes optical features generated when the light source emits light to the lens of the target glasses.

The embodiment corresponding to the above FIG. 1 can be combined, the VR device provided by the embodiment of the present disclosure performs image acquisition on a user wearing glasses. When image acquisition is performed through a camera on the VR device, an LED lamp on the VR device is turned on, and the LED lamp emits light to a lens of target glasses, so that an image containing optical features generated by the light on the lens of the glasses can be acquired, and the optical features can include features such as a light spot, a lens texture, and lens thickness information formed by the light on the lens of the target glasses.

In an embodiment, a VR device includes a first lens barrel and a second lens barrel. The first lens barrel and the second lens barrel are respectively provided with an image acquisition device and at least one light source, and the image acquisition device can be a camera.

In step S320, inputting input data including an image to be processed to the glasses diopter identification model to obtain the glasses diopter of the target glasses.

The glasses diopter identification model is obtained by training a first preset model with a plurality of first type of training samples, the first type of training samples include lens images of preset glasses, the lens images include optical features generated when the light source emits light to the preset glasses, and the training samples carry corresponding glasses diopters. The preset glasses can include glasses with different styles and different diopters.

In an embodiment, the image to be processed may be an image corresponding to one lens in the target glasses, so that the glasses diopter of the lens may be obtained through the glasses diopter identification model. The image to be processed may also be images corresponding to two lenses of the target glasses, that is, a first lens image and a second lens image, and the first lens image and the second lens image may be respectively input into the glasses diopter identification model to respectively obtain the glasses diopters of the two lenses of the target glasses. Or, the image to be processed may also be an image obtained by splicing the first lens image and the second lens image of the target glasses, and the spliced image is input to the glasses diopter identification model, so that the glasses diopters of the two lenses of the target glasses can be obtained at the same time. It should be noted that, in the embodiment of the present disclosure, the spliced image may be directly used as an input of the glasses diopter identification model, and the input data at least including the spliced image may also be used as an input of the glasses diopter identification model.

In the embodiments, based on the description of the above embodiments, the optical features generated on the lens by the light emitted from the light source can reflect the glasses diopter. For example, the optical features may include one or more of light spots, lens texture, and lens thickness information, which may be used as a basis for identifying the glasses diopter. Thus, the image to be processed may contain one or more of the features of light spot, lens texture and lens thickness information. In order to more accurately identify the glasses diopter, in the embodiment, the image to be processed may include the light spot and the lens texture at the same time, or the image to be processed may include the light spot, the lens texture and the lens thickness information at the same time.

In addition, during the process of training the first preset model with the first type of the training samples to obtain the glasses diopter identification model, the training samples can contain one or more of the features such as light spots, lens textures, lens thickness information and the like, and each training sample carries the glasses diopter of the corresponding lens. The first preset model can be a neural network model and other models, the first preset model is trained with a training sample, and a glasses diopter identification model can be obtained after training is completed.

According to the glasses diopter identification method provided by the embodiment of the disclosure, the glasses diopter of the target glasses is obtained by acquiring the image to be processed and taking the input data containing the image to be processed as the input of the glasses diopter identification model. Since the image to be processed is an image obtained by performing image acquisition on the lens of the target glasses under the irradiation of the light source, the image to be processed includes optical features generated when the light source emits light to the lens of the target glasses, and the optical features generated on the lens by the light emitted by the light source can reflect the glasses diopter. Therefore, when the glasses diopter identification is carried out on the image to be processed through the glasses diopter identification model, since the glasses diopter identification model is obtained through training with the optical features generated when the light source emits light to the lenses of the preset glasses and training samples carrying corresponding glasses diopters, the glasses diopter of the target glasses in the image to be processed can be well identified through the glasses diopter identification model.

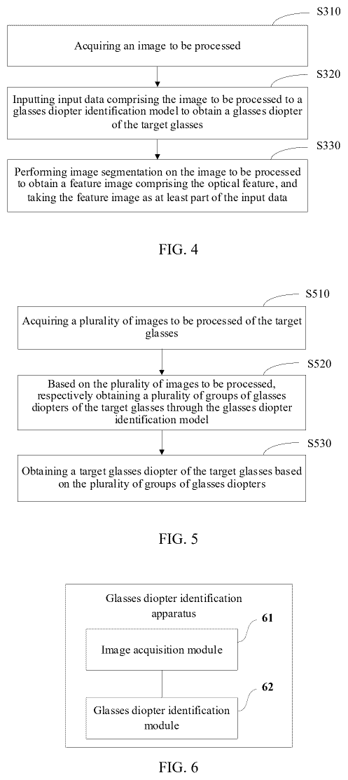

In an embodiment provided by the present disclosure, based on the above embodiment, the glasses diopter identification method provided by the present disclosure, as shown in FIG. 4, may further include the following steps:
in step S330, performing image segmentation on the image to be processed to obtain a feature image including the optical feature, and taking the feature image as at least part of the input data.

In an embodiment, since the optical feature includes one or more of the light spot, the lens texture and the lens thickness information, for example, when the feature image includes the lens image and the light spot image, the image to be processed is input into the image segmentation model to obtain the lens image and the light spot image. The image segmentation model is obtained by training a second preset model with a second type of training sample, and the second preset model can be a neural network model or other models. The second type of training sample includes an image obtained by synthesizing different user images and different preset feature images, and the preset feature images include pre-marked light spot areas and pre-marked lens areas. The user image is a face image at least containing the eyes of the user.

Therefore, the feature image and the image to be processed are input into the glasses diopter identification model as input data, namely, the original image of the target glasses and the feature image of the original image are input into the glasses diopter identification model, so that the accuracy rate of the glasses diopter identification model for identifying the glasses diopter of the target glasses can be improved, and the glasses diopter of the target glasses can be identified more accurately.

It should be noted that, for the training process of the image segmentation model and the manner of synthesizing the training samples, reference may be made to the description of the above embodiment, and details are not repeated here.

In the embodiment provided by the present disclosure, if the feature image includes a lens image and a light spot image and the lens image includes a lens texture, then the image to be processed, the lens image, and the light spot image may be spliced in a channel dimension, and the spliced image is used as an input of the glasses diopter identification model. The glasses texture is not easy to be separated, so that the lens image carrying the glasses texture can be obtained after the light spot is separated from the image to be processed. The image to be processed, the lens image and the light spot image are spliced in the channel dimension, and the image to be processed, the lens image and the light spot image are associated to form an integral image, so that the target glasses diopter can be more accurately identified by the glasses diopter identification model.

In the embodiment provided by the present disclosure, when the VR device provided by the above embodiment is used for performing image acquisition of target glasses, images of two lenses of the target glasses may be obtained, that is, a first lens image and a second lens image of the target glasses are obtained. In the embodiment, the first lens image and the second lens image can be spliced in the horizontal direction, and the spliced images are used as the images to be processed, so that when the images to be processed are identified through the glasses diopter identification model to obtain the glasses diopter of the target glasses, the glasses diopter of the first lens and the glasses diopter of the second lens of the target glasses can be identified at the same time, and the identification effect is better. Since there is a certain correlation between the glasses diopters of the left and right eyes of a human body in general, when the first lens image and the second lens image are spliced and the spliced image is input to the glasses diopter identification model, a more accurate identification effect can be achieved.

In the embodiment provided by the disclosure, the image to be processed can be acquired by the image acquisition device positioned on the VR device. The image acquisition device at least includes first image acquisition device and second image acquisition device, the first image acquisition device is configured to acquire an image of a first lens in the target glasses, the second image acquisition device is configured to acquire an image of a second lens in the target glasses, and at least one light source is arranged on the VR device. The VR device provided by the embodiment of the disclosure acquires the image of the target glasses on the premise of obtaining the authorization permission of the user. In a case where the target user wearing VR device is detected, whether the target user is wearing glasses or not can be determined. If the target user is not wearing glasses, image capture of the target user's eyes may be stopped. If the target user is wearing the glasses, light is emitted to the lenses of the target glasses through a light source on the VR device, and image acquisition is carried out on the lenses of the target glasses through image acquisition device on the VR device. The glasses may be the target glasses in the embodiments. In this way, by determining whether the user is wearing glasses before carrying out image acquisition and then determining whether to perform image acquisition, it is possible to improve the efficiency of identification, avoid the problem of poor user experience caused by emitting light to the user and collecting images when the user is not wearing them, and can also improve the identification efficiency of glasses degrees.

Based on the foregoing embodiment, in another embodiment provided by the present disclosure, as shown in FIG. 5, the method may further include the following steps:
in step S510, acquiring a plurality of images to be processed of the target glasses.

In step S520, based on the plurality of images to be processed, respectively obtaining a plurality of groups of glasses diopters of the target glasses through the glasses diopter identification model.

Each group of glasses diopter includes a first lens diopter and a second lens diopter of the target glasses.

In step S530, obtaining a target glasses diopter of the target glasses based on the plurality of groups of glasses diopters.

In the embodiment, in order to improve the accuracy of the glasses diopter of the target glasses, a plurality of images of the target glasses may be continuously acquired, processed in the manner of the above embodiment, and input into the glasses diopter identification model to obtain the glasses identification diopter corresponding to each image to be processed, so as to obtain a plurality of groups of glasses diopters of the target glasses. The glasses diopter of the target glasses can be determined by averaging, and the glasses diopter of the target glasses is more accurate. In addition, when the target glasses diopter of the target glasses is obtained based on the plurality of groups of glasses diopters, abnormal values in the plurality of groups of glasses diopters can be eliminated, and then the glasses diopters of the target glasses can be obtained by averaging the glasses diopters of the rest groups, so that the obtained result is more accurate.

It is understood that, before the technical solutions disclosed in the embodiments of the present disclosure are used, the user should be informed of the type, the use range, the use scene, etc. of the personal information related to the present disclosure in a proper manner according to the relevant laws and regulations and obtain the authorization of the user.

For example, in response to receiving a user's active request, prompt information is sent to the user to explicitly prompt the user that the requested operation to be performed would require acquisition and use of personal information to the user. Thus, the user can autonomously select whether to provide personal information to software or hardware such as an electronic device (including a wearing device such as a VR device), an application, a server, or a storage medium that performs the operation of the technical solution of the present disclosure, according to the prompt information.

As an alternative but non-limiting implementation manner, in response to receiving an active request from the user, the manner of sending the prompt information to the user may be, for example, a pop-up window, and the prompt information may be presented in a text in the pop-up window. In addition, a selection control for the user to select “agree” or “disagree” to provide personal information to an electronic device (such as a VR device) can be carried in the popup.

It is understood that the above notification and user authorization process is only illustrative and is not intended to limit the implementation of the present disclosure, and other ways of satisfying the relevant laws and regulations may be applied to the implementation of the present disclosure.

The embodiment of the disclosure provides a glasses diopter identification apparatus, which can be a VR device. FIG. 6 is a schematic block diagram of functional modules of a glasses diopter identification apparatus according to an exemplary embodiment of the present disclosure. As shown in FIG. 6, the glasses diopter identification apparatus includes:
an image acquisition module 61 configured to acquire an image to be processed; the image to be processed is obtained by performing image acquisition on a lens of target glasses under irradiation of a light source, and the image to be processed includes an optical feature generated when the light source emits light to the lens of the target glasses; and
a glasses diopter identification module 62 configured to input data including the image to be processed to a glasses diopter identification model to obtain a glasses diopter of the target glasses.

In another embodiment provided by the present disclosure, the apparatus further include:
an image segmentation module configured to perform image segmentation on the image to be processed to obtain a feature image including the optical feature, and taking the feature image as at least part of the input data.

In another embodiment provided by the present disclosure, the optical feature includes one or a combination of: light spots, lens texture, or lens thickness information.

In another embodiment provided by the present disclosure, the apparatus further includes:
a training module configured to train a first preset model with a plurality of first type of training samples to obtain the glasses diopter identification model, wherein the first type of training samples include lens images of preset glasses, the lens images include optical feature generated when a light source emits light to the preset glasses, and the training samples carry corresponding glasses diopters.

In another embodiment provided by the present disclosure, the feature image includes a lens image and a light spot image, and the image segmentation module is specifically configured to:
input the image to be processed into an image segmentation model to obtain the lens image and the light spot image; the image segmentation model is obtained by training a second preset model with a second type of training sample, wherein the second type of training sample includes an image obtained by synthesizing different user images and different preset feature images, and the preset feature images include pre-marked light spot areas and pre-marked lens areas.

In another embodiment provided by the present disclosure, the feature image includes a lens image and a light spot image, the lens image includes a lens texture, and glasses diopter identification module is specifically configured to:
splice the image to be processed, the lens image and the light spot image in a channel dimension, and taking the spliced image as an input of the glasses diopter identification model.

The lens image includes a first lens image and a second lens image, and the apparatus further includes:
a splicing processing module configured to splice the first lens image and the second lens image in a channel dimension, splice the first lens image and the second lens image in a horizontal direction, and take the spliced image as an input of the glasses diopter identification model.

In another embodiment provided by the present disclosure, the apparatus further includes:
a lens image acquisition module configured to acquire a first lens image and a second lens image of the target glasses; and
an image splicing module configured to splice the first lens image and the second lens image in a horizontal direction, and take the spliced image as the image to be processed.

In another embodiment provided by the present disclosure, the image to be processed is acquired by an image acquisition device located on a VR device; wherein the image acquisition device includes at least a first image acquisition device configured to capture an image of a first lens of the target eyewear and a second image acquisition device configured to capture an image of a second lens of the target eyewear, the VR device having at least one light source disposed thereon.

In another embodiment provided by the present disclosure, the apparatus further includes:
a glasses wearing determining module configured to in a case where the target user is detected to be wearing the VR device, determine whether the target user is wearing the target glasses; and
an image acquisition module, configured to in a case where the target user is wearing the target glasses, emit light to the lenses of the target glasses through the light source on the VR device, and acquire the images of the lenses of the target glasses by the image acquisition device on the VR device.

In another embodiment provided by the present disclosure, the apparatus further includes:
an images to be processed acquisition module configured to acquire a plurality of images to be processed of the target glasses; and
an images to be processed identification module configured to, based on the plurality of images to be processed, respectively obtain a plurality of groups of glasses diopters of the target glasses through the glasses diopter identification model; wherein each group of glasses diopter includes a first lens diopter and a second lens diopter of the target glasses; and
a target glasses diopter acquisition module configured to obtain a target glasses diopter of the target glasses based on the plurality of groups of glasses diopters.

Since the apparatus corresponds to the above method, its description is specifically referred to the description corresponding to the above method embodiments, which will not be repeated here.

According to the glasses diopter identification apparatus provided by the embodiment of the disclosure, the glasses diopter of the target glasses is obtained by acquiring the image to be processed and taking the input data containing the image to be processed as the input of the glasses diopter identification model. Since the image to be processed is an image obtained by performing image acquisition on the lens of the target glasses under the irradiation of the light source, the image to be processed includes optical features generated when the light source emits light to the lens of the target glasses, and the optical features generated on the lens by the light emitted by the light source can reflect the glasses diopter. Therefore, when the glasses diopter identification is carried out on the image to be processed through the glasses diopter identification model, since the glasses diopter identification model is obtained through training with the optical features generated when the light source emits light to the lenses of the preset glasses and training samples carrying corresponding glasses diopters, the glasses diopter of the target glasses in the image to be processed can be well identified through the glasses diopter identification model.

The embodiment of the disclosure provides an electronic device, which can be the above VR device and includes: at least one processor; a memory for storing instruction that is executable by the at least one processor; wherein the at least one processor is configured to execute the instructions to implement the above method of the embodiment of the present disclosure.

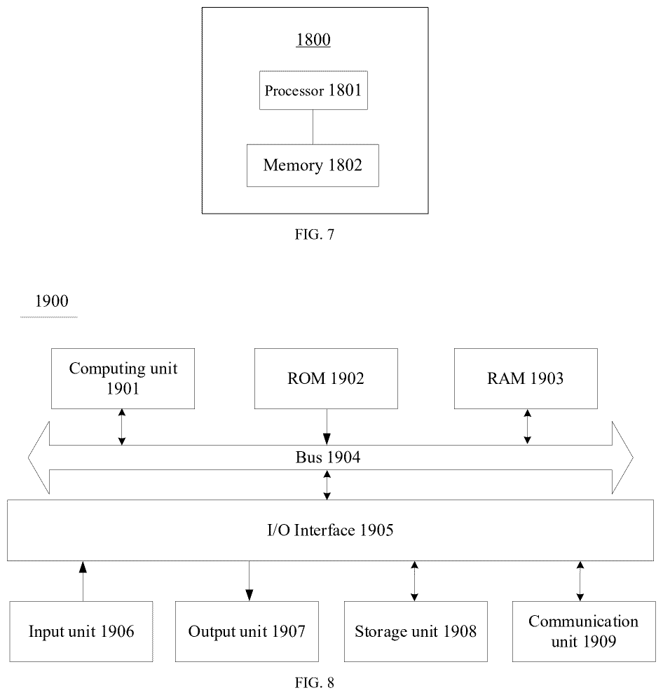

FIG. 7 is a schematic structural diagram of an electronic device provided by an exemplary embodiment of the present disclosure. As shown in FIG. 7, the electronic device 1800 includes at least one processor 1801 and a memory 1802 coupled to the processor 1801, and the processor 1801 can execute the corresponding steps in the above method disclosed in the embodiment of the present disclosure.

The processor 1801 can also be called a central processing unit (CPU), which can be an integrated circuit chip with signal processing capability. Each step in the above method disclosed in the embodiment of the present disclosure can be completed by an integrated logic circuit of hardware or an instruction in the form of software in the processor 1801. The processor 1801 can be a general processor, a digital signal processing (DSP), an application specific integrated circuit (ASIC), a field-programmable gate array (FPGA) or other programmable logic devices, discrete gate or transistor logic devices, and discrete hardware components. The general processor can be a microprocessor or the processor can be any conventional processor, etc. The steps of the method disclosed in combination with the embodiment of the present disclosure can be directly embodied as being completed by a hardware decoding processor, or being completed by a combination of hardware and software modules in the decoding processor. The software module can be located in the memory 1802, such as random access memory, flash memory, read-only memory, programmable read-only memory or electrically erasable programmable memory, registers and other mature storage media in the field. The processor 1801 reads the information in the memory 1802 and completes the steps of the above method in combination with its hardware.

In addition, when various operations/processes according to the present disclosure are implemented by software and/or firmware, programs constituting the software can be installed from a storage medium or a network to a computer system with a dedicated hardware structure, such as the computer system 1900 shown in FIG. 8, and the computer system can perform various functions, including such functions as those described above, when various programs are installed. FIG. 8 is a structural block diagram of a computer system provided by an exemplary embodiment of the present disclosure.

The computer system 1900 is intended to represent various forms of digital electronic computer equipment, such as laptop computers, desktop computers, workstations, personal digital assistants, servers, blade servers, mainframe computers, and other suitable computers. Electronic devices can also represent various forms of mobile devices, such as personal digital processing, cellular phones, smart phones, wearable devices and other similar computing devices. The components shown herein, their connections and relationships, and their functions are only examples, and are not intended to limit the implementation of the present disclosure described and/or claimed herein.

As shown in FIG. 8, a computer system 1900 includes a computing unit 1901, which can perform various appropriate actions and processes according to a computer program stored in a read-only memory (ROM) 1902 or a computer program loaded from a storage unit 1908 into a random access memory (RAM) 1903. In the RAM 1903, various programs and data required for the operation of the computer system 1900 can also be stored. A computing unit 1901, a ROM 1902 and a RAM 1903 are connected to each other through a bus 1904. An input/output (I/O) interface 1905 is also connected to the bus 1904.

A number of components in the computer system 1900 are connected to the I/O interface 1905, including: an input unit 1906, an output unit 1907, a storage unit 1908 and a communication unit 1909. The input unit 1906 may be any type of device that can input information to the computer system 1900, and the input unit 1906 may receive input numeric or character information and generate key signal input related to user settings and/or function control of the electronic device. The output unit 1907 may be any type of device capable of presenting information, and may include but not limited to a display, a speaker, a video/audio output terminal, a vibrator and/or a printer. The storage unit 1908 may include, but is not limited to, a magnetic disk and an optical disk. The communication unit 1909 allows the computer system 1900 to exchange information/data with other devices through a network such as the Internet, and may include but not limited to a modem, a network card, an infrared communication device, a wireless communication transceiver and/or a chipset, such as a Bluetooth™ device, a WiFi device, a WiMax device, a cellular communication device and/or the like.

The computing unit 1901 may be various general and/or special processing components with processing and computing capabilities. Some examples of the computing unit 1901 include, but are not limited to, a central processing unit (CPU), a graphics processing unit (GPU), various specialized artificial intelligence (AI) computing chips, various computing units running machine learning model algorithms, a digital signal processor (DSP), and any suitable processor, controller, microcontroller, etc. The computing unit 1901 performs the various methods and processes described above. For example, in some embodiments, the above-described method disclosed in the embodiments of the present disclosure can be implemented as a computer software program tangibly embodied in a machine-readable medium, such as the storage unit 1908. In some embodiments, part or all of the computer program may be loaded and/or installed on the electronic device 1900 via the ROM 1902 and/or the communication unit 1909. In some embodiments, the computing unit 1901 may be configured to perform the above-mentioned method disclosed in the embodiments of the present disclosure by any other suitable means (for example, by means of firmware).

Embodiments of the present disclosure also provide a computer-readable storage medium, wherein instructions in the computer-readable storage medium, when executed by a processor of an electronic device, enable the electronic device to perform the above method disclosed in embodiments of the present disclosure.

The computer-readable storage medium in the embodiment of the present disclosure may be a tangible medium, which may contain or store a program for use by or in combination with an instruction execution system, device or device. The computer-readable storage medium may include, but is not limited to, an electronic, magnetic, optical, electromagnetic, infrared, or semiconductor system, device or equipment, or any suitable combination of the above. More specifically, the computer-readable storage medium may include an electrical connection based on one or more lines, a portable computer disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination of the above.

The computer-readable medium may be included in the electronic device; or it can exist alone without being assembled into the electronic equipment.

Embodiments of the present disclosure also provide a computer program including instructions that, when executed by a processor, cause the processor to perform the above-mentioned method disclosed in embodiments of the present disclosure.

Embodiments of the present disclosure also provide a computer program product, including instructions, wherein the instructions, when executed by a processor, cause the processor to perform the above method disclosed in embodiments of the present disclosure.

In embodiments of the present disclosure, computer program codes for performing the operations of the present disclosure can be written in one or more programming languages or their combinations, including but not limited to object-oriented programming languages such as Java, Smalltalk, C++, and conventional procedural programming languages such as “C” or similar programming languages. The program code can be completely executed on the user's computer, partially executed on the user's computer, executed as an independent software package, partially executed on the user's computer and partially executed on a remote computer, or completely executed on a remote computer or server. In the case involving a remote computer, the remote computer may be connected to a user computer through any kind of network, including a local area network (LAN) or a wide area network (WAN), or may be connected to an external computer.

The flowcharts and block diagrams in the drawings illustrate the architecture, functions and operations of possible implementations of systems, methods and computer program products according to various embodiments of the present disclosure. In this regard, each block in the flowchart or block diagram may represent a module, a program segment, or a part of code that contains one or more executable instructions for implementing specified logical functions. It should also be noted that in some alternative implementations, the functions noted in the blocks may occur in a different order than those noted in the drawings. For example, two blocks shown in succession may actually be executed substantially in parallel, and they may sometimes be executed in the reverse order, depending on the functions involved. It should also be noted that each block in the block diagrams and/or flowcharts, and combinations of blocks in the block diagrams and/or flowcharts, can be implemented by a dedicated hardware-based system that performs specified functions or operations, or by a combination of dedicated hardware and computer instructions.

The modules, components or units described in the embodiments of the present disclosure can be realized by software or hardware. Among them, the name of the module, component or unit does not constitute the limitation of the module, component or unit itself in some cases.

The functions described above herein may be at least partially performed by one or more hardware logic components. For example, without limitation, exemplary hardware logic components that can be used include: Field Programmable Gate Array (FPGA), Application Specific Integrated Circuit (ASIC), Application Specific Standard Product (ASSP), System on Chip (SOC), Complex Programmable Logic Device (CPLD) and so on.

The above descriptions are only some embodiments of the present disclosure and explanations of the applied technical principles. It should be understood by those skilled in the art that the disclosure scope involved in this disclosure is not limited to the technical scheme formed by the specific combination of the above technical features, but also covers other technical schemes formed by any combination of the above technical features or their equivalent features without departing from the above disclosure concept. For example, the above features are replaced with (but not limited to) technical features with similar functions disclosed in this disclosure.

Although some specific embodiments of the present disclosure have been described in detail through examples, it should be understood by those skilled in the art that the above examples are only for illustration and are not intended to limit the scope of the present disclosure. It should be understood by those skilled in the art that the above embodiments can be modified without departing from the scope and spirit of the present disclosure. The scope of the present disclosure is defined by the appended claims.

1. A glasses diopter identification method, comprising:
acquiring an image to be processed; the image to be processed is obtained by performing image acquisition on a lens of target glasses under irradiation of a light source, and the image to be processed comprises an optical feature generated when the light source emits light to the lens of the target glasses; and
inputting input data comprising the image to be processed to a glasses diopter identification model to obtain a glasses diopter of the target glasses.

2. The method of claim 1, further comprises:
performing image segmentation on the image to be processed to obtain a feature image comprising the optical feature, and taking the feature image as at least part of the input data.

3. The method of claim 1, wherein the optical feature comprises one or a combination of: light spots, lens texture, or lens thickness information.

4. The method of claim 1, further comprises:
training a first preset model with a plurality of first type of training samples to obtain the glasses diopter identification model, wherein the first type of training samples comprise lens images of preset glasses, the lens images comprise optical feature generated when the light source emits light to the preset glasses, and the training samples carry corresponding glasses diopters.

5. The method of claim 2, wherein the feature image comprises a lens image and a light spot image, and the performing image segmentation on the image to be processed comprises:
inputting the image to be processed into an image segmentation model to obtain the lens image and the light spot image; the image segmentation model is obtained by training a second preset model with a second type of training sample, wherein the second type of training sample comprises an image obtained by synthesizing different user images and different preset feature images, and the preset feature images comprise pre-marked light spot areas and pre-marked lens areas.

6. The method of claim 2, wherein the feature image comprises a lens image and a light spot image, the lens image comprises a lens texture, and the inputting input data comprising the image to be processed to a glasses diopter identification model comprises:
splicing the image to be processed, the lens image and the light spot image in a channel dimension, and taking the spliced image as an input of the glasses diopter identification model.

7. The method of claim 1, further comprises:
acquiring a first lens image and a second lens image of the target glasses; and
splicing the first lens image and the second lens image in a horizontal direction, and taking the spliced image as the image to be processed.

8. The method of claim 1, wherein the image to be processed is acquired by an image acquisition device located on a VR device; wherein the image acquisition device comprises at least a first image acquisition device configured to capture an image of a first lens of the target eyewear and a second image acquisition device configured to capture an image of a second lens of the target eyewear, the VR device having at least one light source disposed thereon.

9. The method of claim 8, further comprises:
in response to that the target user is detected to be wearing the VR device, determining whether the target user is wearing the target glasses;
in response to that the target user is wearing the target glasses, emitting light to the lenses of the target glasses through the light source on the VR device, and acquiring the images of the lenses of the target glasses by the image acquisition device on the VR device.

10. The method of claim 1, further comprises:
acquiring a plurality of images to be processed of the target glasses;
based on the plurality of images to be processed, respectively obtaining a plurality of groups of glasses diopters of the target glasses through the glasses diopter identification model; wherein each group of glasses diopter comprises a first lens diopter and a second lens diopter of the target glasses; and
obtaining a target glasses diopter of the target glasses based on the plurality of groups of glasses diopters.

11. (canceled)

12. An electronic device, comprising:
at least one processor;
a memory for storing instruction that is executable by the at least one processor;
wherein the at least one processor is configured to execute the instructions to implement a glasses diopter identification method, comprising:
acquiring an image to be processed; the image to be processed is obtained by performing image acquisition on a lens of target glasses under irradiation of a light source, and the image to be processed comprises an optical feature generated when the light source emits light to the lens of the target glasses; and
inputting input data comprising the image to be processed to a glasses diopter identification model to obtain a glasses diopter of the target glasses.

13. A non-transient computer-readable storage medium, wherein instructions in the computer-readable storage medium, when executed by a processor of an electronic device, enable the electronic device to perform a glasses diopter identification method, comprising:
acquiring an image to be processed; the image to be processed is obtained by performing image acquisition on a lens of target glasses under irradiation of a light source, and the image to be processed comprises an optical feature generated when the light source emits light to the lens of the target glasses; and
inputting input data comprising the image to be processed to a glasses diopter identification model to obtain a glasses diopter of the target glasses.

14. The electronic device of claim 12, further comprises:
performing image segmentation on the image to be processed to obtain a feature image comprising the optical feature, and taking the feature image as at least part of the input data.

15. The electronic device of claim 12, wherein the optical feature comprises one or a combination of: light spots, lens texture, or lens thickness information.

16. The electronic device of claim 12, further comprises:
training a first preset model with a plurality of first type of training samples to obtain the glasses diopter identification model, wherein the first type of training samples comprise lens images of preset glasses, the lens images comprise optical feature generated when the light source emits light to the preset glasses, and the training samples carry corresponding glasses diopters.

17. The electronic device of claim 14, wherein the feature image comprises a lens image and a light spot image, and the performing image segmentation on the image to be processed comprises:
inputting the image to be processed into an image segmentation model to obtain the lens image and the light spot image; the image segmentation model is obtained by training a second preset model with a second type of training sample, wherein the second type of training sample comprises an image obtained by synthesizing different user images and different preset feature images, and the preset feature images comprise pre-marked light spot areas and pre-marked lens areas.

18. The electronic device of claim 14, wherein the feature image comprises a lens image and a light spot image, the lens image comprises a lens texture, and the inputting input data comprising the image to be processed to a glasses diopter identification model comprises:
splicing the image to be processed, the lens image and the light spot image in a channel dimension, and taking the spliced image as an input of the glasses diopter identification model.

19. The electronic device of claim 12, further comprises:
acquiring a first lens image and a second lens image of the target glasses; and
splicing the first lens image and the second lens image in a horizontal direction, and taking the spliced image as the image to be processed.

20. The electronic device of claim 12, wherein the image to be processed is acquired by an image acquisition device located on a VR device; wherein the image acquisition device comprises at least a first image acquisition device configured to capture an image of a first lens of the target eyewear and a second image acquisition device configured to capture an image of a second lens of the target eyewear, the VR device having at least one light source disposed thereon.

21. The electronic device of claim 20, further comprises:
in response to that the target user is detected to be wearing the VR device, determining whether the target user is wearing the target glasses;
in response to that the target user is wearing the target glasses, emitting light to the lenses of the target glasses through the light source on the VR device, and acquiring the images of the lenses of the target glasses by the image acquisition device on the VR device.
